(19) United States
(12) Patent Application Publication
(10) Pub. No.: US 2009/0204017 A1
(43) Pub. Date: Aug. 13, 2009

(54) Uterine activity monitoring using impedance plethysmography

(76) Inventor: Ashit M. Pandit, Laurel, MD (US)

Correspondence Address: ROGER M. RATHBUN, 13 MARGARITA COURT, HILTON HEAD ISLAND, SC, 29926, US

(21) Appl. No.: 12/069,649

(22) Filed: Feb. 12, 2008

(51) Int. Cl. A61B 5/053

(52) U.S. Cl. 600/547

(57) ABSTRACT

A method and system for monitoring the uterine activity of a patient. A pair of electrodes are position in contact with the skin of the patient in the abdominal area. An electrical signal is introduced into the patient by the electrodes and a return signal is received and detected by the same electrodes. The return signal is analyzed and processed to produce a signal that is indicative of the uterine activity of the patient. As such, the system analyzes the electrical impedance and which changes with contractions or other uterine activity of the patient. As such, only two relatively light electrodes are utilized and both transmit and receive the electrical signal to determine changes in the impedance between the electrodes which is interpreted to be indicative of the uterine activity.

### FIELD OF THE INVENTION

The present invention relates to a system for measuring uterine activity, and, more particularly, to the use of impedance plethsymography to measure the uterine activity.

### BACKGROUND OF THE INVENTION

The uterine activity of pregnant women is routinely monitored near the onset of labor. At the present, such uterine activity is carried out by the use of monitoring transducers, cables, attachment belts and the like and which are generally cumbersome, heavy and create a burden on the patient during the difficult period of time.

For example, one current means of monitoring uterine activity is through the use of an external pressure measuring device called a tocodynamometer. The transducers for such monitors are heavy and bulky. In addition it is often necessary to reposition the transducer on the abdomen of the patient because the sensing interface needs to be perpendicular to the applied force to accurately measure the pressure changes. Due to the large size of the typical transducer, the transducer itself presses into the patient's abdomen causing discomfort and normally the transducer requires an attachment belt to position and retain the transducer in the desired position.

Accordingly, it would be advantageous to have a system and a method that could carry out the monitoring of uterine activity with smaller, simpler devices that are applied with light electrical leads and thus minimize the discomfort to the patient. It would also be advantageous to have a uterine activity monitor that can remain at one position on the abdomen of the patient and not need to be continually re-located.

### BRIEF SUMMARY OF THE INVENTION

Therefore, with the present invention, there is provided a method and system for monitoring the uterine activity of a pregnant patient. In the method aspect of the present invention there is a method that comprises positioning a pair of electrodes in contact with the skin of a patient in the abdominal area. An AC electrical signal is applied through the electrodes whereby the AC signal enters the patient and a return signal is received by the electrodes. The uterine activities, such as contractions, are sensed by changes in the impedance seen on the abdomen of the patient and are evidenced by an amplitude modulation of the AC signal. The return signal is then processed by demodulation and the resultant analog signal can then be converted to digital signals which can be displayed by a monitor with a visual display that provides a visual form of the uterine activity based on those change in impedance. The AC signal can be a pulsed or continuous wave signal to the uterus and, in an exemplary embodiment, the frequency of the AC signals can be from about 50 kHz to about 1.5 MHz.

There is also a system that carries out the procedure of the present invention and which includes pair of electrodes that are adapted to be positioned in contact with the abdomen of the patient. Again, the electrical signal is directed inwardly, and a return signal is sensed by the electrodes and the received return signals are processed to extract and provide signals indicative of the change in impedance of the uterus and, in an exemplary embodiment, a visual display of that information representative of uterine activity.

Accordingly, the electrodes and the associated wires can be lightweight and not impose a burden on the patient. Further, since the present invention relies on a change in impedance of the mother's abdomen, the electrodes do not need to be repositioned as the monitoring continues over time.

These and other features and advantages of the present invention will become more readily apparent during the following detailed description taken in conjunction with the drawings herein.

### BRIEF DESCRIPTION OF THE DRAWINGS

FIG. 1 is a schematic view of the uterine activity monitor of the present invention.

### DETAILED DESCRIPTION OF THE INVENTION

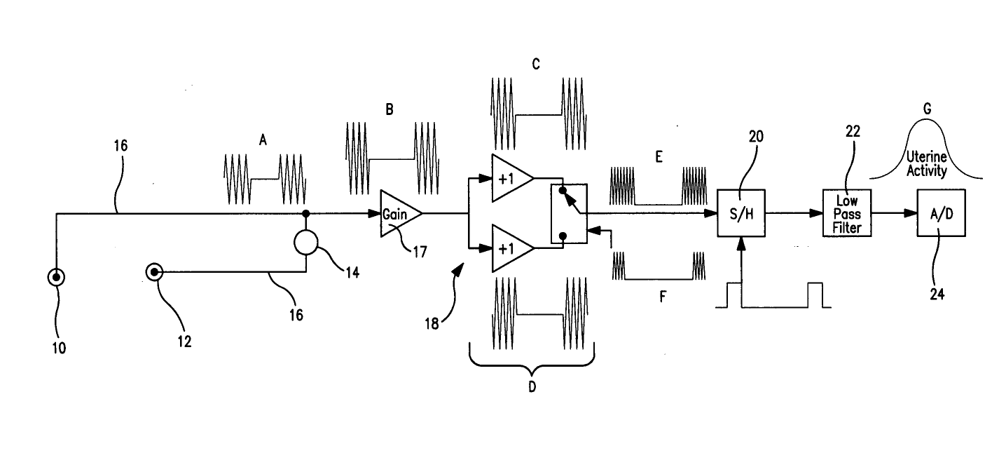

Referring now to FIG. 1, there is shown a schematic view of the present invention and wherein there are a pair of electrodes 10, 12 that are adapted to be positioned in contact with the skin of the patient in the abdominal area.

An electrical signal source 14 is present in order to provide an electrical signal to the electrodes 10, 12 to thereafter transmit that electrical signal inwardly toward the uterus of the patient. In the exemplary embodiment, that electrical signal is an alternating current (AC) signal having a frequency range of from about 50 kHz to about 1.5 MHz to the patient. That electrical signal can be transmitted to the electrodes 10, 12 by relatively light wires 16 so as to not burden the patient. Accordingly, both the electrodes 10, 12 and the wires 16 are all light and do not impose a weight burden on the abdomen of the patient.

The electrodes introduce the signal into the patient and return signals are, in turn, received by the same electrodes 10, 12. Those return signals contain information as to the impedance of the uterus and changes in that impedance is indicative of the activity of the uterus i.e. changes in the electrical impedance of the patient's impedance occur during contractions and thus those changes in impedance are indicative of uterine activity. In effect, the change in the impedance of the uterus causes amplitude modulation of the applied AC signal.

The return signals from the patient are shown as A in FIG. 1 and the signals are then passed through the gain amplifier 17 where the gain is increased to reproduce the signals shown as B. The signals are then passed through a demodulator 18 producing signals C and D and hence signals E and F where the demodulated signals are indicative of the change in impedance of the uterus.

The signals are then integrated with respect to time, at function 20 and passed through a low pass filter 22. The resulting signals are a wave form shown as G and those signals can thereafter be digitized by an analog to digital converter 24 to produce digital signals indicative of the change of impedance, thereby indicative of the uterine activity of the patient.

As such the present system and method monitor the impedance of the uterus and that impedance provides an indication of the uterine activity of the patient.

Those skilled in the art will readily recognize numerous adaptations and modifications which can be made to the of the present invention which will result in an improved device and method of using the same, yet all of which will fall within the scope and spirit of the present invention as defined in the following claims. Accordingly, the invention is to be limited only by the following claims and their equivalents.

What is claimed is:

1. A method for monitoring uterine activity of a patient, the method comprising the steps of:
(a) positioning a pair of electrodes against the skin of a patient;
(b) applying a voltage through the electrodes to enter the patient;
(c) sensing a return signal to the electrodes; and
(d) using the return signal to derive an output indicative of changes in the impedance between the electrodes.

2. The method of claim 1 wherein the step of applying a voltage through the electrodes comprises applying a continuous wave AC voltage to the patient.

3. The method of claim 2 wherein the step of applying a voltage through the electrodes comprises applying an AC voltage having a frequency of between about 50 kHz to 1.5 MHz.

4. The method of claim 1 wherein the step of using the return signals to derive an output further includes converting the signals received by the electrodes to digital signals indicative of the uterine activity of the patient.

5. The method of claim 1 wherein the step of using the return signals to derive and output includes the steps of demodulating the signals, integrating the demodulated signals with respect to time and displaying signals indicative of the uterine activity.

6. The method of claim 1 wherein the method further includes providing a visual display and the step of converting the signals comprises displaying the signal indicative of uterine activity on the visual display.

7. A system for monitoring the uterine activity of a patient, the system comprising;
a pair of electrodes adapted to be positioned on the skin of the patient;
a source of electricity for supplying an AC electrical signal to the electrodes,
circuitry for a receiving a signal from the electrodes to determine the change in impedance between the electrodes positioned on the patient.

8. The system of claim 7 wherein the system includes a display for displaying the change in impedance between the electrodes.

9. The system of claim 7 wherein the circuitry demodulates the signals from the electrodes.

10. The system of claim 9 wherein the demodulated signals are digitized.

11. The system of claim 7 wherein the source of electricity provides an AC voltage having a frequency from about 50 kHz to 1.5 MHz.
